(19) United States
(12) Patent Application Publication
(10) Pub. No.: US 2006/0003985 A1
(43) Pub. Date: Jan. 5, 2006

(54) Enhancement of sleep with t-type calcium channel antagonists

(76) Inventors: John J. Renger, North Wales, PA (US); Kenneth S. Koblan, Carversville, PA (US)

Correspondence Address: MERCK AND CO., INC, P O BOX 2000, RAHWAY, NJ, 07065-0907, US

(21) Appl. No.: 10/531,504

(22) PCT Filed: Oct. 15, 2003
(86) PCT No.: PCT/US03/32892

§ 371 Date: Apr. 13, 2005

Related U.S. Application Data
(60) Provisional application No. 60/419,203, filed on Oct. 17, 2002.

(51) Int. Cl. A61K 31/554; A61K 31/455

(52) U.S. Cl. 514/211.07; 514/355

(57) ABSTRACT

A T-type calcium channel antagonist is useful, alone or in combination with other agents, for inducing sleep and enhancing and improving the quality of sleep, in particular by increasing sleep efficiency and augmenting sleep maintenance.

### BACKGROUND OF THE INVENTION

Plasma membrane calcium channels are members of a diverse family of channel proteins. Calcium channels are membrane-spanning, multi-subunit proteins that allow controlled entry of Ca2+ ions into cells from the extracellular fluid. Cells throughout the animal kingdom, and at least some bacterial, fungal and plant cells, possess one or more types of calcium channel. The most common type of calcium channel is voltage dependent. All “excitable” cells in animals, such as neurons of the central nervous system (CNS), peripheral nerve cells and muscle cells, including those of skeletal muscles, cardiac muscles, and venous and arterial smooth muscles, have voltage-dependent calcium channels. “Opening” of a voltage-dependent channel to allow an influx of Ca2+ ions into the cells requires a depolarization to a certain level of the potential difference between the inside of the cell bearing the channel and the extracellular environment bathing the cell. The rate of influx of Ca2+ into the cell depends on this potential difference.

Multiple types of calcium channels have been identified in mammalian cells from various tissues, including skeletal muscle, cardiac muscle, lung, smooth muscle and brain. A major member of this family is the L-type calcium channels, which bind the familiar classes of calcium channel blockers (dihydropyridines such as nifedipine, phenylalkylamines such as verapamil, and benzothiazepines such as diltiazem). Additional classes of plasma membrane calcium channels, referred to as T, N, P, Q and R, are found in other tissues. These membrane proteins are homologous both to each other and to the extended family of voltage-gated plasma membrane ion channels and are distinguished by current kinetics, holding potential sensitivity and sensitivity to calcium channel agonists and antagonists.

The “T-type” (or “low voltage-activated”) calcium channels, so named because their openings are of briefer duration (T=transient) than the longer (L=long-lasting) openings of the L-type calcium channels, have a lower conductance and so admit less calcium when they are open than L-type channels. “T-type” (low voltage-activated) channels are affected by molecules that transiently activate at negative potentials and are highly sensitive to changes in resting potential. The L, N, P and Q-type channels activate at more positive potentials (high voltage activated) and display diverse kinetics and voltage-dependent properties.

There are three subtypes of T-type calcium channels that have been molecularly, pharmacologically, and electrophysiologically identified from various warm blooded animals including rat [J. Biol. Chem. 276(6) 3999-4011 (2001); Eur J Neurosci 11(12):4171-8(1999); reviewed in Cell Mol Life Sci 56(7-8):660-9 (1999)]. These subtypes have been termed α1G, α1H, and α1I. The molecular properties of these channels demonstrate that the amino acid sequences are between 60-70% identical. The electrophysiological characterization of these individual subtypes has revealed differences in their voltage-dependent activation, inactivation, deactivation and steady-state inactivation levels and their selectivities to various ions such as barium (J. Biol. Chem. 276(6) 3999-4011 (2001)). Pharmacologically, these subtypes also have differing sensitivities to blockade by ionic nickel. These channel subtypes are also expressed in various forms due to their ability to undergo various splicing events during their assembly (J. Biol. Chem. 276(6) 3999-4011 (2001)).

Although sleep is necessary for survival, its precise homeostatic contribution is unknown. Sleep is not a uniform state, but rather involves several stages characterized by changes in the individual's EEG. A non rapid eye movement (NREM) type (75 to 80% of total sleep time) ranges in depth through stages 1 to 4 (deepest level). Stage 1 sleep is drowsiness, in which the EEG displays a lower voltage, more mixed frequencies and deterioration of alpha rhythm relative to the EEG when the individual is awake. In stage 2, background activity similar to that of stage 1 is experienced, with bursts of slightly higher frequency “sleep spindles” and sporadic higher amplitude slow wave complexes. The third and fourth stages of sleep display increasing high amplitude slow wave activity. The separate sleep stage in which the individual undergoes rapid eye movement (REM) occupies the remainder of the sleep time and occurs 5 to 6 times during a normal nights sleep. REM sleep is characterized by a lower voltage, higher frequency EEG and other characteristics similar to those which occur when the individual is awake, whereas the other four sleep stages are categorized as NREM sleep.

Individuals vary widely in their requirements for sleep, which is influenced by a number of factors including their current emotional state. The natural aging process is associated with changes in a variety of circadian and diurnal rhythms. Age-related changes in the timing and structure of sleep are surprisingly common problems for older people, and are often associated with significant morbidity. With advancing age, the total amount of sleep tends to shorten. Stage 4 can decrease or disappear and sleep may become more fragmented and interrupted. Evaluation of sleep patterns in elderly people shows that the timing of sleep is also phase advanced, especially in women. This tendency to go to sleep and wake up earlier is very frustrating to older people who feel that they are out of step with the rest of the world. In addition, the quality of sleep in the elderly is diminished with a marked reduction in slow wave sleep, a reduction in the deep stages of sleep (especially stage 4), fragmentation of REM sleep and more frequent awakenings. Similarly, non-elderly people may exhibit disturbances in the normal sleep process. These changes in the structure of sleep have been correlated to more frequent napping, decreased daytime alertness and declining intellectual function and cognitive ability. Deprivation of REM sleep has been suggested to interfere with the memory consolidation involved in learning skills through repetitive activity, and slow wave sleep has been implicated as being important in consolidation of events into long term memory. Likewise, decreases in the length of REM stages of sleep may be associated with a decrease in cognitive function and learning, especially diminished retention of memory.

Sleep disorders generally involve disturbances of sleep that affect a subject's ability to fall and/or stay asleep, and involve sleeping too little, too much or resulting in abnormal behavior associated with sleep.

Numerous compounds are employed in the art to facilitate normal sleep and to treat sleep disorders and sleep disturbances, including e.g., sedatives, hypnotics, anxiolytics, antipsychotics, antianxiety agents, minor tranquilizers, melatonin agonists and antagonists, melatonergic agents, benzodiazepines, barbiturates, 5HT-2 antagonists, and the like. Similarly, physical methods have been employed to treat patients with sleep disorders such as the use of light therapy or the application of modulated electrical signals to selected nerves or nerve bundles.

Nevertheless, the known therapeutic regimens suffer from numerous problems, including residual sleepiness and related effects in daytime function, impairment of memory, potential for addiction, rebound insomnia, “REM rebound” which may be associated with increased dream intensity and the occurrence of nightmares, seizure induction, interaction with other medicines and alcohol to cause severe impairment and other health problems, and the like. Accordingly, a more physiological way to enhance sleep would be highly desirable.

### SUMMARY OF THE INVENTION

The present invention is directed to the use of a compound which has the ability to antagonize T-type calcium channels for enhancing or improving sleep quality, in particular by increasing sleep efficiency and augmenting sleep maintenance, as well as for preventing and treating sleep disorders and sleep disturbances, in a warm-blooded animal. The present invention provides a method for enhancing or improving sleep quality and increasing sleep efficiency and sleep maintenance in a warm-blooded animal comprising the administration of a T-type calcium channel antagonist. The present invention further provides a pharmaceutical composition for enhancing or improving sleep quality and increasing sleep efficiency and sleep maintenance.

### BRIEF DESCRIPTION OF THE DRAWINGS

FIG. 1 shows that a T-type calcium channel antagonist reduced the entries into wakefulness relative to vehicle and had a significant reduction in sleep fragmentation.

FIG. 2 shows that a T-type calcium channel antagonist reduced the wake duration relative to vehicle and had a significant decrease in the duration of wakefulness.

### DESCRIPTION OF THE INVENTION

The present invention is directed to the use of a compound which has the ability to antagonize T-type calcium channels for enhancing or improving sleep quality as well as preventing and treating sleep disorders and sleep disturbances in a warm-blooded animal. In addition, the present invention provides a method for enhancing sleep quality, improving sleep quality, increasing sleep efficiency and augmenting sleep maintenance. Further, the present invention provides a method for preventing and treating sleep disorders and sleep disturbances in a warm-blooded animal which comprising the administration of a T-type calcium channel antagonist. The present invention further provides a pharmaceutical composition for enhancing or improving sleep quality, augmenting sleep and increasing sleep efficiency and sleep maintenance.

The present method of using a T-type calcium channel antagonist provides beneficial outcomes in a subject which may be correlated to enhancement in sleep quality. Use of a T-type calcium channel antagonist in accordance with the present invention further provides the following: an increase in the value which is calculated from the time that a subject sleeps divided by the time that a subject is attempting to sleep; a decrease in sleep latency (the time it takes to fall asleep); a decrease in difficulties in falling asleep; a decrease in the number of awakenings during sleep; a decrease in nocturnal arousals; a decrease in the time spent awake following the initial onset of sleep; an increase in the total amount of sleep; an increase the amount and percentage of REM sleep; an increase in the duration and occurrence of REM sleep; a reduction in the fragmentation of REM sleep; a decrease in the amount and percentage of slow-wave (i.e. stage 3 or 4) sleep; an increase in the amount and percentage of stage 2 sleep; an enhancement of EEG-delta activity during sleep; a decrease in nocturnal arousals, especially early morning awakenings; an increase in daytime alertness; a reduction in daytime drowsiness; an increased satisfaction with the intensity of sleep; and increased sleep maintenance. Secondary outcomes which may be provided by the present invention include enhanced cognitive function, enhanced memory and increased memory retention.

Another embodiment of the present invention is directed to a method for the treatment, control, amelioration or reduction of risk of a sleep disorder and sleep disturbance including sleep problems associated with insomnia, hypersomnia, interrupted sleep, sleep apnea, narcolepsy, nocturnal myoclonus, REM sleep interruptions, jet-lag, shift workers' sleep disturbances, dyssomnias, night terror, insomnias associated with depression or with emotional/mood disorders, as well as sleep walking and enuresis, and sleep disorders which accompany aging. Sleep disorders and sleep disturbances are generally characterized by difficulty in initiating or maintaining sleep or in obtaining restful or enough sleep. Another embodiment of the present invention is directed to a method for the treatment, control, amelioration or reduction of risk of a sleep-associated disorder.

Another embodiment of the present invention is directed to a method for the treatment, control, amelioration or reduction of risk of a condition associated with circadian rhythmicity as well as mental and physical disorders associated with travel across time zones and with rotating shift-work schedules. In addition, certain drugs may also cause reductions in REM sleep as a side effect and the present invention may be used to correct those types of sleeping disorders as well. The present invention would also be of benefit in the treatment of syndromes such as fibromyalgia which are manifested by non-restorative sleep and muscle pain or sleep apnea which is associated with respiratory disturbances during sleep. It will be clear to one skilled in the art that the present invention is not limited to just sleep disorders and sleep disturbances, but is applicable to a wide variety of conditions which result from a diminished quality of sleep.

Another embodiment of the present invention is directed to a method for the treatment, control, amelioration or reduction of risk of a disease or disorder where abnormal oscillatory activity occurs in the brain, including depression, migraine, neuropathic pain, Parkinson's disease, psychosis and schizophrenia, as well as diseases or disorders where there is abnormal coupling of activity, particularly through the thalamus.

By the term “T-type calcium channel antagonist” is meant any exogenously administered compound or agent that directly or indirectly antagonizes the activity of T-type (low voltage-activated) calcium channels in an animal, in particular, a human.

The T-type calcium channel antagonist may be peptidal or non-peptidal in nature, however, the use of a non-peptidal T-type calcium channel antagonist is preferred. In addition, for convenience the use of an orally active T-type calcium channel antagonist is preferred. In an alernate embodiment, the T-type calcium channel antagonist inhibits t-type calcium channels at night or during the sleep cycle, especially in the first half of the night or of the sleep cycle, and even more especially in the first few hours following sleep onset, or alternatively in the period immediately preceding sleep onset.

In an embodiment of the present invention the T-type calcium channel antagonist is a selective antagonist of a T-type calcium channel.

In an embodiment of the present invention the compound that is a selective antagonist of a T-type calcium channel possesses a selectivity for the T-type calcium channel relative to the L-type calcium channel of at least 5 fold as measured by the ratio of IC₅₀ for the T-type calcium channel to the IC₅₀ for the L-type calcium channel as evaluated by the voltage-clamp assay. In a further embodiment, the selective antagonist of a T-type calcium channel possesses a selectivity for the T-type calcium channel relative to the L-type calcium channel of at least 10 fold as measured by the ratio of IC₅₀ for the T-type calcium channel to the IC₅₀ for the L-type calcium channel as evaluated by the voltage-clamp assay. In a further embodiment, the selective antagonist of a T-type calcium channel possesses a selectivity for the T-type calcium channel relative to the L-type calcium channel of at least 50 fold as measured by the ratio of IC₅₀ for the T-type calcium channel to the IC₅₀ for the L-type calcium channel as evaluated by the voltage-clamp assay. In a further embodiment, the selective antagonist of a T-type calcium channel possesses a selectivity for the T-type calcium channel relative to the L-type calcium channel of at least 100 fold as measured by the ratio of IC₅₀ for the T-type calcium channel to the IC₅₀ for the L-type calcium channel as evaluated by the voltage-clamp assay. In a further embodiment, the selective antagonist of a T-type calcium channel possesses a selectivity for the T-type calcium channel relative to the L-type calcium channel of at least 200 fold as measured by the ratio of IC₅₀ for the T-type calcium channel to the IC₅₀ for the L-type calcium channel as evaluated by the voltage-clamp assay. In a further embodiment, the selective antagonist of a T-type calcium channel possesses a selectivity for the T-type calcium channel relative to the L-type calcium channel of at least 500 fold as measured by the ratio of IC₅₀ for the T-type calcium channel to the IC₅₀ for the L-type calcium channel as evaluated by the voltage-clamp assay.

In a further embodiment of the present invention the compound that is a selective antagonist of a T-type calcium channel possesses a selectivity for the T-type calcium channel of at least 5 fold as measured by the ratio of IC₅₀ for the T-type calcium channel to the IC₅₀ for at least one other receptor, enzyme or ion channel as evaluated by the T-type calcium channel antagonist voltage-clamp assay relative to the binding assay for the receptor, enzyme or ion channel.

In a further embodiment of the present invention the compound that is a selective antagonist of a T-type calcium channel possesses a selectivity for the T-type calcium channel of at least 10 fold as measured by the ratio of IC₅₀ for the T-type calcium channel to the IC₅₀ for at least one other receptor, enzyme or ion channel as evaluated by the T-type calcium channel antagonist voltage-clamp assay relative to the binding assay for the receptor, enzyme or ion channel.

In a further embodiment of the present invention the compound that is a selective antagonist of a T-type calcium channel possesses a selectivity for the T-type calcium channel of at least 5 fold as measured by the ratio of IC₅₀ for the T-type calcium channel to the IC₅₀ for the neurokinin-1 receptor as evaluated by the T-type calcium channel antagonist voltage-clamp assay relative to the binding assay for the neurokinin-1 receptor (U.S. Pat. No. 5,484,886 and U.S. Pat. No. 5,525,712).

In an embodiment of the present invention, the antagonist of a T-type calcium channel possesses a selectivity for the α1G subtype T-type calcium channel relative to the α1H subtype and/or α1I subtype T-type calcium channel of at least 10 fold as measured by the ratio of IC₅₀ for the α1G subtype T-type calcium channel to the IC₅₀ for the α1H subtype and/or all subtype T-type calcium channel as evaluated by the voltage-clamp assay.

In an embodiment of the present invention, the antagonist of a T-type calcium channel possesses a selectivity for the α1H subtype T-type calcium channel relative to the α1G subtype and/or α1I subtype T-type calcium channel of at least 10 fold as measured by the ratio of IC₅₀ for the α1H subtype T-type calcium channel to the IC₅₀ for the α1G subtype and/or all subtype T-type calcium channel as evaluated by the voltage-clamp assay.

In an embodiment of the present invention, the antagonist of a T-type calcium channel possesses a selectivity for the all subtype T-type calcium channel relative to the α1G subtype and/or α1H subtype T-type calcium channel of at least 10 fold as measured by the ratio of IC₅₀ for the all subtype T-type calcium channel to the IC₅₀ for the α1G subtype and/or α1H subtype T-type calcium channel as evaluated by the voltage-clamp assay.

In an embodiment of the present invention the antagonist of a T-type calcium channel possesses an IC₅₀ for binding to the T-type calcium channel of 1 μM or less as evaluated by the T-type calcium channel antagonist voltage-clamp assay. In another embodiment of the present invention the antagonist of a T-type calcium channel possesses an IC₅₀ for binding to the T-type calcium channel of 500 nM or less as evaluated by the T-type calcium channel antagonist voltage-clamp assay. In another embodiment of the present invention the antagonist of a T-type calcium channel possesses an IC₅₀ for binding to the T-type calcium channel of 100 nM or less as evaluated by the T-type calcium channel antagonist voltage-clamp assay. In another embodiment of the present invention antagonist of a T-type calcium channel possesses an IC₅₀ for binding to the T-type calcium channel of 50 nM or less as evaluated by the T-type calcium channel antagonist voltage-clamp assay. In another embodiment of the present invention the antagonist of a T-type calcium channel possesses an IC₅₀ for binding to the T-type calcium channel of 1 nM or less as evaluated by the T-type calcium channel antagonist voltage-clamp assay.

In an embodiment of the present invention the T-type calcium channel antagonist is a CNS-penetrant T-type calcium channel antagonist and is able to enter the brain and/or central nervous system with sufficient concentration to have a therapeutic effect. In a further embodiment of the present invention the CNS-penetrant T-type calcium channel antagonist is a compound that exhibits sufficient concentration in the brain and/or central nervous system to have therapeutic efficacy upon oral adminstration.

In an embodiment of the present invention the T-type calcium channel antagonist has an onset of action of 45-60 minutes. In another embodiment of the present invention the T-type calcium channel antagonist has an onset of action of 30-45 minutes. In another embodiment of the present invention the T-type calcium channel antagonist has an onset of action of 15-30 minutes. In another embodiment of the present invention the T-type calcium channel antagonist has an onset of action of less than 15 minutes.

In an embodiment of the present invention the T-type calcium channel antagonist has a pharmacological half life (T½ life) of ultra short duration. In another embodiment of the present invention the T-type calcium channel antagonist has a pharmacological half life (T½ life) of short duration. In another embodiment of the present invention the T-type calcium channel antagonist has a pharmacological half life (T½ life) of intermediate duration. In another embodiment of the present invention the T-type calcium channel antagonist has a pharmacological half life (T½ life) of long duration. In another embodiment of the present invention the T-type calcium channel antagonist has a pharmacological half life (T½ life) of at least about 2 hours duration, but less than about 6 hours duration. In another embodiment of the present invention the T-type calcium channel antagonist has a pharmacological half life (T½ life) of at least about 3 hours duration, but less than about 5 hours duration.

In accordance with the present invention, use of a T-type calcium channel antagonist is particularly useful for alterering the arousal levels of the organism to promote sleep, and specifically to enhance sleep maintenance and reduce sleep fragmentation. This would be especially useful in returning the fragmented sleep patterns observed in the elderly humans to more normal sleep patterns which are observed in the young adult humans. Use of a T-type calcium channel antagonist is further useful for the promotion of sleep with a reduced suppression of REM sleep. This property of the T-type calcium channel antagonists used in accordance with the present invention is different from the effects seen in the benzodiazepines and non-benzodiazepine GABA_{A} receptor agonist compounds (e.g. diazepam or zolpidem) which are commonly used to treat sleep disorders in the clinical setting. Furthermore, GABA receptor modulators potentially exhibit undesirable activity with respect to respiratory, withdrawal, tolerance, or drug interactions.

The T-type calcium channel antagonist may be used alone or in combination with other T-type calcium channel antagonists or with other agents which are known to be beneficial in the enhancement of sleep efficiency. The T-type calcium channel antagonist and the other agent may be co-administered, either in concomitant therapy or in a fixed combination. For example, the T-type calcium channel antagonist may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the T-type calcium channel antagonist may be administered in conjunction with the use of physical methods such as with light therapy or electrical stimulation.

For use in medicine, the salts of the compounds employed in this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following: Acetate, Benzenesulfonate, Benzoate, Bicarbonate, Bisulfate, Bitartrate, Borate, Bromide, Calcium, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Subacetate, Succinate, Sulfate, Sulfonate, Tannate, Tartrate, Teoclate, Tosylate, Triethiodide and Valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

T-type (Low-voltage activated) calcium channels are fully disclosed in e.g., U.S. Pat. No. 5,618,720, U.S. Pat. No. 5,686,241, U.S. Pat. No. 5,710,250,U.S. Pat. No. 5,726,035, U.S. Pat. No. 5,792,846, U.S. Pat. No. 5,846,757, U.S. Pat. No. 5,851,824, U.S. Pat. No. 5,874,236, U.S. Pat. No. 5,876,958, U.S. Pat. No. 6,013,474, U.S. Pat. No. 6,057,114, U.S. Pat. No. 6,096,514, EP 1042468, WO 99/28342, and J. Neuroscience, 19(6):1912-1921 (1999).

The identification of a compound as a T-type calcium channel antagonist may be readily determined without undue experimentation by methodology well known in the art, including the “T-type Calcium (Ca²⁺) Antagonist Voltage-Clamp Assay”. In a typical experiment ion channel function from HEK 293 cells expressing the T-type channel alpha-1H is recorded to determine the activity of compounds in blocking that the T-type channel alpha-1H. By appropriate modifications readily apparent to one skilled in the art, this protocol may also be used to determine the activity of compounds in blocking the G or I subtypes of T-type channels. In this T-type calcium (Ca²⁺) antagonist voltage-clamp assay calcium currents are elicited from the resting state of the human alpha-1H calcium channel as follows. Sequence information for T-type (Low-voltage activated) calcium channels are fully disclosed in e.g., U.S. Pat. No. 5,618,720, U.S. Pat. No. 5,686,241, U.S. Pat. No. 5,710,250,U.S. Pat. No. 5,726,035, U.S. Pat. No. 5,792,846, U.S. Pat. No. 5,846,757, U.S. Pat. No. 5,851,824, U.S. Pat. No. 5,874,236, U.S. Pat. No. 5,876,958, U.S. Pat. No. 6,013,474, U.S. Pat. No. 6,057,114, U.S. Pat. No. 6,096,514, WO 99/28342, and J. Neuroscience, 19(6):1912-1921 (1999). Cells expressing the t-type channels were grown in H3D5 growth media which comprised DMEM, 6% bovine calf serum (HYCLONE), 30 micromolar Verapamil, 200 microgram/ml Hygromycin B, 1× Penicillin/Streptomycin. Glass pipettes are pulled to a tip diameter of 1-2 micrometer on a pipette puller. The pipettes are filled with the intracellular solution and a chloridized silver wire is inserted along its length, which is then connected to the headstage of the voltage-clamp amplifier. Trypsinization buffer was 0.05% Trypsin, 0.53 mM EDTA. The extracellular recording solution consists of (mM): 130 mM NaCl, 4 mM KCl, 1 mM MgCl2, 2 mM CaCl₂, 10 mM HEPES, 30 Glucose, pH 7.4. The internal solution consists of (mM): 135 mM CsMeSO4, 1 MgCl2, 10 CsCl, 5 EGTA, 10 HEPES, pH 7.4, or 135 mM CsCl, 2 MgCl2, 3 MgATP, 2 Na2ATP, 1 Na2GTP, 5 EGTA, 10 HEPES, pH 7.4. Upon insertion of the pipette tip into the bath, the series resistance is noted (acceptable range is between 1-4 megaohm). The junction potential between the pipette and bath solutions is zeroed on the amplifier. The cell is then patched, the patch broken, and, after compensation for series resistance (>=80%), the voltage protocol is applied while recording the whole cell Ca2+ current response. Voltage protocols: (1)-80 mV holding potential every 20 seconds pulse to −20 mV for 40 msec duration; the effectiveness of the drug in inhibiting the channel is measured directly from measuring the reduction in peak current amplitude initiated by the voltage shift from −80 mV to −20 mV; (2). −100 mV holding potential every 15 seconds pulse to −20 mV for 40 msec duration; the effectiveness of the drug in inhibiting the channel is measured directly from measuring the reduction in peak current amplitude initiated by the shift in potential from −100 mV to −30 mV. The difference in block at the two holding potentials was used to determine the effect of drug at differing levels of inactivation induced by the level of resting state potential of the cells.

After obtaining control baseline calcium currents, extracellular solutions containing increasing concentrations of a test compound are washed on. Once steady state inhibition at a given compound concentration is reached, a higher concentration of compound is applied. % inhibition of the peak inward control Ca2+ current during the depolarizing step to −20 mV is plotted as a function of compound concentration.

The intrinsic T-type calcium channel antagonist activity of a compounds which may be used in the present invention may be determined by these assays.

The term “therapeutically effective amount” shall mean that amount of a drug or pharmaceutical agent that will elicit the biological or medical response of a tissue, system, animal or human that is being sought by a researcher or clinician.

Accordingly, the present invention includes within its scope the use of a T-type calcium channel antagonist, alone or in combination with other agents, for the prevention or treatment of sleep disorders and sleep disturbances in a warm-blooded animal. For the purposes of this disclosure, a warm-blooded animal is a member of the animal kingdom which includes but is not limited to mammals and birds. The preferred mammal for purposes of this invention is human.

The subject treated in the present methods is generally a mammal, preferably a human being, male or female, in whom antagonism of T-type calcium channel activity is desired. In the present invention, it is preferred that the subject mammal is a human. Although the present invention is applicable both old and young people, it would find greater application in elderly people. Further, although the invention may be employed to enhance the sleep of healthy people, it may be especially beneficial for enhancing the sleep quality of people suffering from sleep disorders or sleep disturbances. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

This particular application of T-type calcium channel antagonists provides unexpected benefit relative to the administration of other agents for the subject indications. T-type calcium channel antagonists which are orally active also have the benefit being able to be administered orally, rather than just intravenously, intraperitoneally or subcutaneously. Although the specific mechanism underlying the present invention is not currently understood, it is likely that inhibition of T-type channels through application of an antagonist leads to a block of bursting in thalamocortical and/or thalamic reticular neurons associated with slow wave sleep. Inhibition of the T-type channels forces these neurons into a state that appears as intermediate to both the bursting “slow wave sleep” and tonically firing “REM/awake states”. Inhibiting T-type channels promotes the amount of time sleep stages 2 and 3 are experienced. Entering REM sleep is critical for the efficacy of sleep and for normal cognitive processes. Altering the sleep structure, through T-type calcium channel inhibition, leads to an enhanced ability to enter into REM from stage 2, thereby minimizing the occurrence of residual sleepiness and poor cognitive performance. The present invention provides a method for altering sleep structure which is completely independent of all other known mechanisms for altering sleep. A principal benefit of T-type inhibition is that it works through a mechanism that appears to specifically counteract sleep disorders, especially sleep disorders that occur in the process of aging, such as decreased REM sleep and fragmented sleep patterns. In contrast to the present invention, existing sleep therapies may suppress REM sleep thereby resulting in daytime drowsiness. Thus, a T-type calcium channel antagonist demonstrates beneficial effects, for example, improved sleep, improved cognition, and/or reduced daytime sleepiness in the elderly, without the problems of REM suppression, addiction, residual sleepiness, and/or cognitive deficits. Accordingly, the present invention provides a mechanism for altering sleep structure, which can counteract deficiencies which occur during normal aging.

The present invention includes within its scope a pharmaceutical composition for enhancing and improving the quality of sleep comprising, as an active ingredient, at least one T-type calcium channel antagonists in association with a pharmaceutical carrier or diluent. Optionally, the active ingredient of the pharmaceutical compositions can comprise another agent in addition to at least one T-type calcium channel antagonist to minimize the side effects or with other pharmaceutically active materials wherein the combination enhances efficacy and minimizes side effects.

The present invention is further directed to a method for the manufacture of a medicament for enhancing sleep, augmenting sleep, improving the quality of sleep and for the treatment of sleep disorders and sleep disturbances in humans comprising combining a compound that is a T-type calcium channel antagonist with a pharmaceutical carrier or diluent.

It will be known to those skilled in the art that there are numerous compounds now being used in an effort to enhance and improve the quality of sleep. Combinations of these therapeutic agents some of which have also been mentioned herein with a T-type calcium channel antagonist will bring additional, complementary, and often synergistic properties to enhance the desirable properties of these various therapeutic agents. In these combinations, the T-type calcium channel antagonist and the therapeutic agents may be independently present in dose ranges from one one-hundredth to one times the dose levels which are effective when these compounds and secretagogues are used singly.

The T-type calcium channel antagonist may be administered in combination with sedatives, hypnotics, anxiolytics, antipsychotics, antianxiety agents, minor tranquilizers, melatonin agonists and antagonists, melatonergic agents, benzodiazepines, barbiturates, 5HT-2 antagonists, and the like, or the T-type calcium channel antagonist may be administered in conjunction with the use of physical methods such as with light therapy or electrical stimulation. For example, to enhance and improve the quality of sleep a T-type calcium channel antagonist may be given in combination with such compounds as: adinazolam, allobarbital, alonimid, alprazolam, amitriptyline, amobarbital, amoxapine, bentazepam, benzoctamine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, as well as admixtures and combinations thereof. To illustrate these combinations, a T-type calcium channel antagonist effective clinically effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that the T-type calcium channel antagonist may be employed with other agents to control sleep disorders and sleep disturbances in depressed patients and/or provide benefit in the prevention or treatment of sleep disorders and sleep disturbances.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. A T-type calcium channel antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection may be formulated according to conventional pharmaceutical practice. Compositions for sublingual administration are also prepared with standard excipients well known in the art.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans, to obtain effective antagonism of T-type calcium channel. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient. The following examples are provided for the purpose of further illustration only and are not intended to be limitations on the disclosed invention.

### EXAMPLE 1

Preclinical Study of a T-Type Calcium Channel Antagonist

In this study, compounds are applied to voltage-clamped cells which express the human subtypes of T-type calcium channel and the percent inhibition of current was determined, as described above. Compounds that were identified as selective inhibitors of the T-type channels and were able to penetrate into the central nervous systems enhanced sleep behaviors in rodent, non-human primate, and/or human mammals. In such studies, the following compounds were able to enhance sleep:

| | | |
|---|---|---|
| Compound: | T-type IC50 | L-type IC50 |

| | | | | |
|---|---|---|---|---|
| Compound B | ˜50 | nM | >˜700 | nM |
| Compound C | ˜200 | nM | >˜2 | uM |
| Compound D | ˜2.7 | uM | ˜19 | uM |

Although these compounds vary widely in their chemical structures, they have a common ability to selectively inhibit T-type calcium channel function and be useful in enhancing sleep.

### EXAMPLE 2

Preclinical Study of the Effects of a T-type Calcium Channel Antagonist on Sleep

This study examined the effects of the T-type calcium channel antagonist Compound A (6-chloro-3,4-dihydro-3-ethyl-4-phenyl-4-ethyl-2(1H)-quinazolinone; PCT WO 93/04047) on sleep by using electrocorticogram (ECoG) or electroencephalographic (EEG) measures to determine the efficacy of the T-type calcium channel antagonistin the altering the states of arousal or sleep.

Eight adult male Sprague Dawley rats (450-550 g; Taconic Farms, Germantown, N.Y.) were subcutaneously implanted with telemetric physiologic monitors (Model F50-EET; Data Sciences International, Arden Hills, Minn.) that were used to simultaneously record both the electrocorticogram (ECoG) and electromyographic (EMG) activities. For placement of ECoG leads, holes slightly smaller than the transmitter lead wire coil diameter, were drilled in the skull 2 mm on either side of midline and 2 mm anterior to the lambda suture and the leads were placed between the skull and underlying dura. EMG leads were placed in the body of a neck muscle. The animals were allowed to recover for at least two weeks prior to recording. Animals were housed individually in plastic cages and were provided water and food ad libitum. Lights were on a 12 hour light: 12 hour dark cycle with lights off at 4:00 a.m. and on at 4:00 p.m. All compounds were dosed approximately 60 minutes prior to lights on. Recordings were started just prior to dosing and were collected for at least 8 hours. Signals were collected simultaneously from the animals with Dataquest software system (Data Sciences International, Arden Hills, Minn.) at 500 Hz and stored on a PC for off-line analysis. Diazepam (Roche) and zolpidem (Sanofi-Synthelabo) were purchased from Myoderm Medical Supply, Norristown, Pa. Both compounds were acutely dissolved in de-ionized water and administered by oral gavage in 2 mL total volume at a final dose of 10 mg/Kg. The study was based on a standard cross-over design with 4 animals receiving compound for one week and the complementary group receiving vehicle (de-ionized water), followed by a week of reversed dosing. All animals were exposed to two days of oral gavage dosing of vehicle prior to drug administration.

Following the completion of data collection, all data were scored with automated sleep stage analysis software, Somnologica (Medcare Co.; Buffalo, N.Y.). Sleep stages were assigned based upon a combination of level of movement within the field of the radio frequency receiver, EMG activity, and EEG frequencies over 10 second epochs. Active wake was assigned to the epoch when movement of the animal was detected within the cage, or when there was an active EMG signal and the EEG consisted of alpha and beta frequency activity. An epoch was scored as light sleep when there was no movement activity, the EMG was moderately active and the EEG consisted of either theta or theta with less than 50% of the epoch showing delta activity. Delta sleep was scored when there was no movement, reduced EMG activity, and the EEG consisted of more than 50% delta wave activity (i.e. 0.5 to 4 Hz). Rapid eye movement (REM) sleep was scored when there was no movement or EMG activity, and the EEG consisted of theta, alpha, and beta activities. Results from the scoring were binned into 30 minute periods following drug administration and the number of entries into each stage and the duration of minutes spent in each stage were calculated. The results for all eight animals were averaged by treatment or vehicle over seven nights and the results were statistically compared based upon Student's t-test for each thirty minute period.

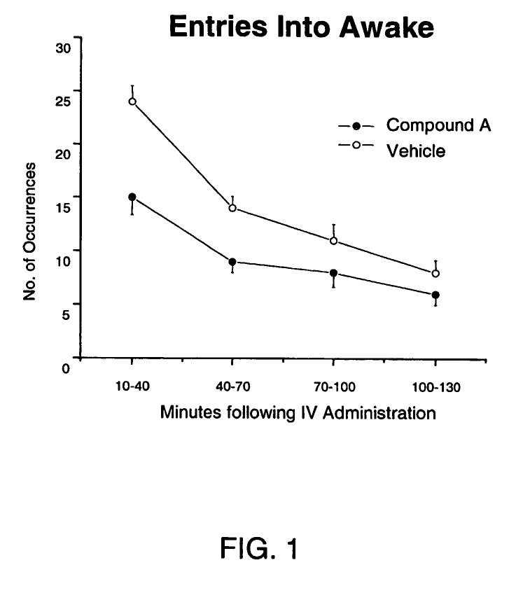

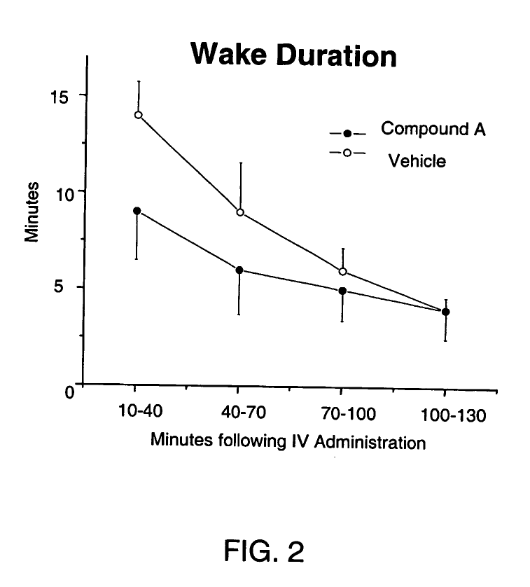

As depicted in FIG. 1, the T-type inhibitor reduced the entries into awake relative to vehicle. This represents a significant reduction in sleep fragmentation as evidenced by a reduction in the number of entries into wakefulness. As depicted in FIG. 2, the T-type inhibitor reduced the wake duration relative to vehicle. This represents a significant decrease in the duration of wakefulness. In accordance with this study, the T-type calcium channel antagonist reduced entry into awake and wake duration and had a beneficial effect on reducing wake time and reducing fragmentation. Thus, the T-type calcium channel antagonist had a beneficial effect on sleep.

### EXAMPLE 3

Clinical Study of a T-type Calcium Channel Antagonist in Healthy Young Adults

In this study, 9 healthy young men (ages 18 to 30 years) who did not suffer sleep complaints are randomly assigned to a sequence of 3 treatment periods. In each period the subjects received a single oral dose of either placebo, 5 mg of T-type calcium channel antagonist or 25 mg T-type calcium channel antagonist once daily for 7 days. Sleep is recorded for 2 nights: a habituation night and a blood sampling night (Days 6 and 7 of study drug administration). This study may be used to assess the efficacy of a T-type calcium channel antagonist on improving sleep efficiency and sleep maintenance and enhancing the quality of sleep in humans.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-32. (canceled)

33. A method for enhancing the quality of sleep in a human patient in need thereof that comprises administering to the patient a therapeutically effective amount of a T-type calcium channel antagonist.

34. A method for augmenting sleep maintenance in a human patient in need thereof that comprises administering to the patient a therapeutically effective amount of a T-type calcium channel antagonist.

35. A method for treating insomnia in a human patient in need thereof that comprises administering to the patient a therapeutically effective amount of a T-type calcium channel antagonist.

36. The method of claim 33 wherein the T-type calcium channel antagonist is a CNS-penetrant T-type calcium channel antagonist.

37. The method of claim 33 wherein the T-type calcium channel antagonist is a selective T-type calcium channel antagonist.

38. The method of claim 37 wherein the T-type calcium channel antagonist possesses a selectivity for the T-type calcium channel relative to the L-type calcium channel of at least 100 fold as measured by the ratio of IC₅₀ for the T-type calcium channel to the IC₅₀ for the L-type calcium channel as evaluated by the voltage-clamp assay.

39. The method of claim 38 wherein the T-type calcium channel antagonist possesses a selectivity for the T-type calcium channel relative to the L-type calcium channel of at least 200 fold as measured by the ratio of IC₅₀ for the T-type calcium channel to the IC₅₀ for the L-type calcium channel as evaluated by the voltage-clamp assay.

40. The method of claim 39 wherein the T-type calcium channel antagonist possesses a selectivity for the T-type calcium channel relative to the L-type calcium channel of at least 500 fold as measured by the ratio of IC₅₀ for the T-type calcium channel to the IC₅₀ for the L-type calcium channel as evaluated by the voltage-clamp assay.

41. The method of claim 33 wherein the T-type calcium channel antagonist possesses a selectivity for the α1G subtype T-type calcium channel relative to the α1H subtype and/or α1I subtype T-type calcium channel of at least 10 fold as measured by the ratio of IC₅₀ for the α1G subtype T-type calcium channel to the IC₅₀ for the α1H subtype and/or α1I subtype T-type calcium channel as evaluated by the voltage-clamp assay.

42. The method of claim 33 wherein the T-type calcium channel antagonist possesses a selectivity for the α1H subtype T-type calcium channel relative to the α1G subtype and/or α1I subtype T-type calcium channel of at least 10 fold as measured by the ratio of IC₅₀ for the α1H subtype T-type calcium channel to the IC₅₀ for the α1G subtype and/or α1I subtype T-type calcium channel as evaluated by the voltage-clamp assay.

43. The method of claim 33 wherein the T-type calcium channel antagonist possesses a selectivity for the α1I subtype T-type calcium channel relative to the α1G subtype and/or α1H subtype T-type calcium channel of at least 10 fold as measured by the ratio of IC₅₀ for the α1I subtype T-type calcium channel to the IC₅₀ for the α1G subtype and/or α1H subtype T-type calcium channel as evaluated by the voltage-clamp assay.

44. The method of claim 33 wherein the T-type calcium channel antagonist possesses an IC₅₀ for binding to the T-type calcium channel of 1 μM or less as evaluated by the T-type calcium channel antagonist voltage-clamp assay.

45. The method of claim 44 wherein the T-type calcium channel antagonist possesses an IC₅₀ for binding to the T-type calcium channel of 500 nM or less as evaluated by the T-type calcium channel antagonist voltage-clamp assay.

46. The method of claim 45 wherein the T-type calcium channel antagonist possesses an IC₅₀ for binding to the T-type calcium channel of 100 nM or less as evaluated by the T-type calcium channel antagonist voltage-clamp assay.

47. The method of claim 46 wherein the T-type calcium channel antagonist possesses an IC₅₀ for binding to the T-type calcium channel of 50 nM or less as evaluated by the T-type calcium channel antagonist voltage-clamp assay.

48. The method of claim 47 wherein the T-type calcium channel antagonist possesses an IC₅₀ for binding to the T-type calcium channel of 1 nM or less as evaluated by the T-type calcium channel antagonist voltage-clamp assay.

49. The method of claim 33 wherein the T-type calcium channel antagonist is an orally active T-type calcium channel antagonist.

50. The method of claim 33 wherein the T-type calcium channel antagonist is orally administered.
